(19) United States
(12) Patent Application Publication
(10) Pub. No.: US 2025/0360335 A1
(43) Pub. Date: Nov. 27, 2025

(54) Multi-Luminaire Gamma Stimulation System With Dual Frequencies

(71) Applicant: Aleddra Inc., Renton, WA (US)

(72) Inventors: Chia-Yiu Maa, Bellevue, WA (US); Li-Jyuan Luo, Taipei City (TW)

(21) Appl. No.: 19/295,635

(22) Filed: Aug. 10, 2025

Related U.S. Application Data
(63) Continuation-in-part of application No. 19/227,439, filed on Jun. 3, 2025.
Application No. 19/227,439 is a continuation-in-part of application No. 19/186,404, filed on Apr. 22, 2025.
Application No. 19/186,404 is a continuation-in-part of application No. 18/626,148, filed on Apr. 3, 2024.
Application No. 18/626,148 is a continuation-in-part of application No. 18/613,079, filed on Mar. 21, 2024.
Application No. 18/613,079 is a continuation-in-part of application No. 18/408,523, filed on Jan. 9, 2024.

(51) Int. Cl. A61N 5/06

(52) U.S. Cl. CPC A61N 5/0622; A61N 2005/0626; A61N 2005/0653

(57) ABSTRACT

A gamma stimulation system comprises a first luminaire with a first controller and a first light source, and a second luminaire with a second controller and a second light source. The first controller operates the first light source at a first frequency F1 (≥60 Hz) generating a first light output. The second controller operates the second light source at a second frequency F2 (>F1) generating a second light output. When the first light output and the second light output are perceived by a subject simultaneously, an invisible visual simulation at a harmonic frequency (HF) equal to |2F1−F2| is induced endogenously in a brain of the subject. By carefully choosing F1 and F2, and subsequently HF, the gamma stimulation system may be used for treating or preventing Alzheimer's disease.

### BACKGROUND

The present disclosure is a continuation-in-part (CIP) of U.S. patent application Ser. No. 19/227,439 filed 3 Jun. 2025, which is a CIP of U.S. patent application Ser. No. 19/186,404, filed 22 Apr. 2025, which is a CIP of U.S. patent application Ser. No. 18/626,148, filed 3 Apr. 2024, which is a CIP of U.S. patent application Ser. No. 18/613,079, filed 21 Mar. 2024, which is itself is a CIP of U.S. patent application Ser. No. 18/408,523, filed 9 Jan. 2024. Contents of aforementioned applications are herein incorporated by reference in their entirety.

### TECHNICAL FIELD

The present disclosure pertains to the field of gamma stimulation system and, more specifically, proposes a gamma stimulation system with dual frequencies.

### DESCRIPTION OF RELATED ART

It has been discovered that by flickering a light at a frequency between 35 Hz to 45 Hz or generating a sound at a similar frequency, it has the effect of stimulating the cells in certain regions of the brain, resulting in using a flicking light or a sound at such a frequency for treating Alzheimer's disease. However, turning on and off a light source at a frequency between 35 Hz to 45 Hz can create visual discomfort in the eyes of a subject. Different approaches have been introduced to overcome this visual discomfort under 40 Hz flickering light.

U.S. patent application Ser. No. 19/227,439 introduces a gamma stimulation apparatus comprising a controller, a first light source, and a second light source. The controller is configured to operate the first light source at a first frequency F1≥60 Hz generating a first light output. Moreover, the controller is configured to operate the second light source at a second frequency F2 (>F1) generating a second light output. When the first light output and the second light output are perceived by a subject simultaneously, an invisible visual simulation at a harmonic frequency (HF) equal to |2F1−F2| is induced endogenously in a brain of the subject.

The gamma stimulation apparatus introduced in U.S. patent application Ser. No. 19/227,439 implicitly restricts embodiments of the apparatus to a single lighting device, i.e., a single luminaire. However, an effective gamma stimulation can be achieved by using two devices where one device operates its light source at a first frequency F1≥60 Hz and the other device operates its light source at a second frequency F2 (>F1), provided that these two devices are sufficiently close to each other such that their light outputs can be perceived by a subject simultaneously. There are no requirements that the two light sources, each operating at a different frequency, must reside in the same device or housing. To overcome this ambiguity, the present disclosure thus expands the gamma stimulation apparatus introduced in U.S. patent application Ser. No. 19/227,439 to a multi-luminaire system where one luminaire operates at F1 and another at F2.

### SUMMARY

In one aspect, the gamma stimulation system comprises a first luminaire with a first controller and a first light source, and a second luminaire with a second controller and a second light source. The first controller is configured to operate the first light source at a first frequency F1 (≥60 Hz) generating a first light output. The second controller is configured to operate the second light source at a second frequency F2 (>F1) generating a second light output. When the first light output and the second light output are perceived by a subject simultaneously, an invisible visual simulation at a harmonic frequency (HF) equal to |2F1−F2| is induced endogenously in a brain of the subject. The first light output is flicker-free for F1≥60 Hz, and the second light output is flicker-free for F2>F1≥60 Hz. Moreover, HF is invisible because it is induced endogenously in the brain of the subject. The gamma stimulation system does not emit such HF. |2F1−F2| refers to the absolute value of the difference of 2F1 and F2. In other words, their difference at 40 Hz and −40 Hz are the same as far as inducing an endogenously gamma stimulation is concerned,

In some embodiments, HF is between 20 Hz and 45 Hz. In practice, HF may be more narrowly chosen to be between 35 Hz and 40 Hz.

In some embodiments, F1 is 80 Hz and F2 is 120 Hz, and thus HF=|2F1−F2|=40 Hz. Similarly, F1 is 80 Hz and F2 is 200 Hz would also generate HF=|2F1−F2|=40 Hz.

In some embodiments, F1 is 65 Hz and F2 is 90 Hz.

In some embodiments, F1 is 70 Hz and F2 is 100 Hz.

In some embodiments, F1 is 75 Hz and F2 is 110 Hz.

In some embodiments, F1 is 85 Hz and F2 is 130 Hz.

In some embodiments, F1 is 90 Hz and F2 is 140 Hz.

In some embodiments, F1 is 120 Hz and F2 is 200 Hz, where |2F1−F2|=240 Hz−200 Hz=40 Hz.

Other research has shown that the slow theta stimulation at 4 Hz and 7 Hz has the effect of triggering memory recall. The present disclosure can be easily devised to support slow theta stimulation. In some embodiments, HF is between 3 Hz and 10 Hz.

In some embodiments, F1 is 60 Hz and F2 is 116 Hz, resulting in HF=4 Hz. In some other embodiments, F1 is 60 Hz and F2 is 124 Hz, also resulting in HF=4 Hz.

In some embodiments, F1 is 60 Hz and F2 is 113 Hz, resulting in HF=7 Hz. In some other embodiments, F1 is 60 Hz and F2 is 127 Hz, also resulting in HF=7 Hz.

For the choice of light sources, light emitting diode (LED) or organic LED (OLED) are preferred because they can operate at a high frequency with accuracy and precision, which is ideal for generating F1, F2, and HF accurately and precisely. Therefore, in some embodiments, the first light source comprises an LED or OLED, and the second light source comprises another LED or OLED. It is to be noted that the present disclosure is not limited to using only LED or DC-driven LED for the light sources. Controllers may be designed to drive AC-driven first and second light sources.

In addition to using LED and OLED as light sources, further measure may be necessary in ensuring the accuracy and precision of HF. This is because inducing an accurate HF (e.g., at 40 Hz) is highly critical in achieving a proper gamma stimulation effect when treating Alzheimer's disease. When HF drifts beyond 40 Hz (e.g., becoming 42 Hz), the effectiveness on treating Alzheimer's disease is greatly reduced or completely vanished. One approach of generating accurate F1 and F2 (and subsequently HF) is using crystal oscillator circuits in the controllers because crystal oscillators are known to generate frequency signal accurately and precisely. Thus, in some embodiments, the first controller comprises a circuit configured to derive F1 internally. Further in some embodiments, the circuit comprises a crystal oscillator circuit.

Similarly, in some embodiments, the second controller comprises a circuit configured to derive F2 internally. Further in some embodiments, the circuit comprises a crystal oscillator circuit.

### BRIEF DESCRIPTION OF THE DRAWINGS

The accompanying drawings are included to aid further understanding of the present disclosure and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 schematically depicts an embodiment of the present disclosure.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of gamma stimulation systems having different form factors.

A gamma stimulation system comprises a first luminaire with a first controller and a first light source, and a second luminaire with a second controller and a second light source. The first controller operates the first light source at a first frequency F1 (≥60 Hz) generating a first light output. The second controller operates the second light source at a second frequency F2 (>F1) generating a second light output. When the first light output and the second light output are perceived by a subject simultaneously, an invisible visual simulation at a harmonic frequency (HF) equal to |2F1−F2| is induced endogenously in a brain of the subject.

### Example Implementations

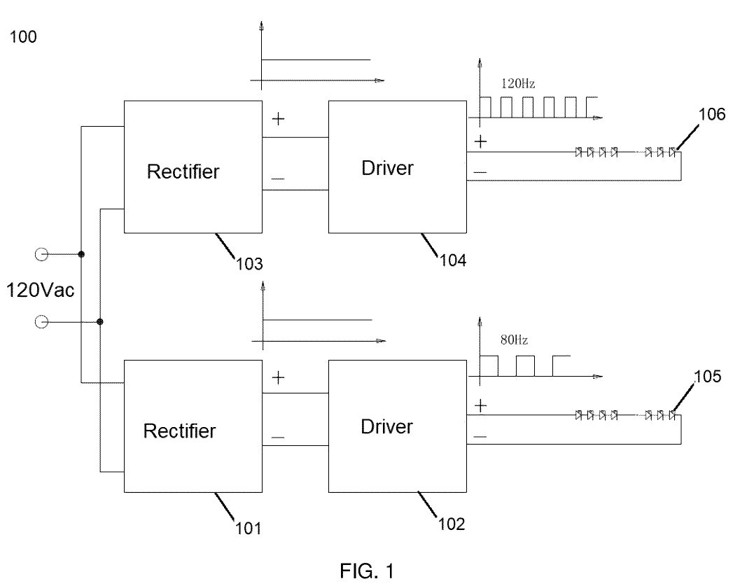

FIG. 1 shows an embodiment of the gamma stimulation system of the present disclosure 100. The first luminaire comprises the first rectifier 101, the first driver 102, and the first light source 105. The first rectifier 101 and the first driver 102 form the first controller. The second luminaire comprises the second rectifier 103, the second driver 104, and the second light source 106. The second rectifier 103 and the second driver 104 form the second controller. The first rectifier 101 converts an external AC power to an internal DC power to power the first driver 102. Similarly, the second rectifier 103 converts an external AC power to an internal DC power to power the second driver 104. The first driver 102 operates the first light source 105 at F1=80 Hz to generate the first light output, and the second driver 104 operates the second light source 106 at F2=120 Hz to generate the second light output. When the first light output and the second light output are perceived by a subject simultaneously, an invisible visual simulation at HF equal to |2F1−F2|=2*80 Hz−120 Hz=40 Hz is induced endogenously in a brain of the subject. The gamma stimulation system does not emit such HF.

Other combinations may be chosen for F1 and F2, such as (65 Hz, 90 Hz), (70 Hz, 100 Hz), (75 Hz, 110 Hz), (85 Hz, 130 Hz), (90 Hz, 140 Hz), (120 Hz, 200 Hz), etc.

The embodiment 200 can be revised to support theta stimulation by choosing F1=60 Hz and F2=116 Hz (or F2=124 Hz), resulting in HF=4 Hz; or F1=60 Hz and F2=113 Hz (or F2=127 Hz), resulting in HF=7 Hz.

The first light source 105 and the second light source 106 are DC-driven LED. The present disclosure is not limited to using only LED or DC-drive LED for light sources. An AC-driven controller may be designed to drive AC-driven first and second light sources, without using any rectifier.

Though not shown in FIG. 1, the first driver 102 and the second driver 104 may comprise crystal oscillator circuit for deriving derive F1 and F2 frequencies, respectively.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A gamma stimulation system, comprising:
a first luminaire with a first controller and a first light source; and
a second luminaire with a second controller and a second light source,
wherein:
the first controller is configured to operate the first light source at a first frequency F1 (≥60 Hz) to generate a first light output,
the second controller is configured to operate the second light source at a second frequency F2 (>F1) to generate a second light output, and
when the first light output and the second light output are perceived by a subject simultaneously, an invisible visual stimulation at a harmonic frequency (HF) equal to |2F1−F2| is induced endogenously in a brain of the subject.

2. The gamma stimulation system of claim 1, wherein HF is between 20 Hz and 45 Hz.

3. The gamma stimulation system of claim 2, wherein F1 is 80 Hz and F2 is 120 Hz.

4. The gamma stimulation system of claim 2, wherein F1 is 65 Hz and F2 is 90 Hz.

5. The gamma stimulation system of claim 2, wherein F1 is 70 Hz and F2 is 100 Hz.

6. The gamma stimulation system of claim 2, wherein F1 is 75 Hz and F2 is 110 Hz.

7. The gamma stimulation system of claim 2, wherein F1 is 85 Hz and F2 is 130 Hz.

8. The gamma stimulation system of claim 2, wherein F1 is 90 Hz and F2 is 140 Hz.

9. The gamma stimulation system of claim 2, wherein F1 is 120 Hz and F2 is 200 Hz.

10. The gamma stimulation system of claim 1, wherein HF is between 3 Hz and 10 Hz.

11. The gamma stimulation system of claim 10, wherein F1 is 60 Hz and F2 is 116 Hz.

12. The gamma stimulation system of claim 10, wherein F1 is 60 Hz and F2 is 124 Hz.

13. The gamma stimulation system of claim 10, wherein F1 is 60 Hz and F2 is 113 Hz.

14. The gamma stimulation system of claim 10, wherein F1 is 60 Hz and F2 is 127 Hz.

15. The gamma stimulation system of claim 1, wherein the first light source comprises a light emitting diode (LED) or organic LED (OLED), and wherein the second light source comprises another LED or OLED.

16. The gamma stimulation system of claim 1, wherein the first controller comprises a circuit configured to derive F1 internally.

17. The gamma stimulation system of claim 16, wherein the circuit comprises a crystal oscillator circuit.

18. The gamma stimulation system of claim 1, wherein the second luminaire comprises a controller comprises a circuit configured to derive F2 internally.

19. The gamma stimulation system of claim 18, wherein the circuit comprises a crystal oscillator circuit.
